(19) United States
(12) Patent Application Publication
(10) Pub. No.: US 2025/0073206 A1
(43) Pub. Date: Mar. 6, 2025

(54) SOLID FORMS OF MESEMBRINE AND THERAPEUTIC USES THEREOF

(71) Applicant: Sensorium Therapeutics, Inc., Boston, MA (US)

(72) Inventors: Xiaoyang Wang, Shanghai (CN); Pengjian Liu, Shanghai (CN)

(21) Appl. No.: 18/705,703

(22) PCT Filed: Oct. 28, 2022
(86) PCT No.: PCT/US2022/048126

§ 371 Date: Apr. 29, 2024

Related U.S. Application Data
(60) Provisional application No. 63/273,728, filed on Oct. 29, 2021.

(51) Int. Cl. A61K 31/404; A61K 47/12; A61K 47/18; A61K 47/20; A61K 47/22; A61K 47/26; A61K 47/28

(52) U.S. Cl. CPC A61K 31/404; A61K 47/12; A61K 47/183; A61K 47/20; A61K 47/22; A61K 47/26; A61K 47/28

(57) ABSTRACT

Solid forms of mesembrine are provided, along with related methods of manufacture. The solid form compositions are useful, for example, in the preparation of pharmaceutical compositions.

### RELATED APPLICATIONS

This application claims the benefit of priority to U.S. Provisional Patent Application No. 63/273,728, filed Oct. 29, 2021.

### TECHNICAL FIELD

This disclosure relates to solid forms of mesembrine, and related therapeutic methods of inhibiting the sodium-dependent serotonin transporter (SERT).

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol are pharmacologically active alkaloids present in Sceletium tortuosum extracts used for treatment of anxiety, stress and mental health conditions.

Natural products obtained from plants of the genus Sceletium contain varying amounts of (−) mesembrine and (+)/(−) mesembrenone. The structure of mesembrine, also known as 3a-(3,4-dimethoxyphenyl)-octahydro-1-methyl-6H-indol-6-one, has been reported by Popelak et al., Naturwiss. 47,156 (1960), and the configuration by P W Jeffs et al., J. Am. Chem. Soc. 91, 3831 (1969). Naturally occurring (−) mesembrine from Sceletium tortuosum has been reported as having serotonin (5-HT) uptake inhibitory activity useful in treating mental health conditions such as mild to moderate depression. Naturally occurring (+)/(−) mesembrenone from Sceletium tortuosum is reported as a potent selective serotonin reuptake inhibitor (Ki=27 nM).

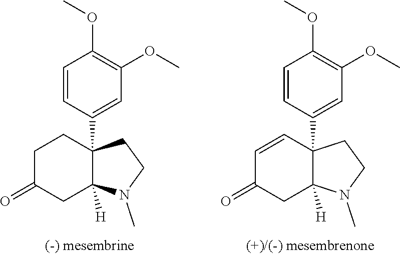 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC]

Polymorphs, solvates and salts of various drugs have been described in the literature as imparting novel properties to the drugs. Organic small drug molecules have a tendency to self-assemble into various polymorphic forms depending on the environment that drives the self-assembly. Heat and solvent mediated effects can also lead to changes that transform one polymorphic form into another.

Identifying which polymorphic form is the most stable under each condition of interest and the processes that lead to changes in the polymorphic form is crucial to the design of the drug manufacturing process in order to ensure that the final product is in its preferred polymorphic form. Different polymorphic forms of an active pharmaceutical ingredient (API) can lead to changes in the drug's solubility, dissolution rate, pharmacokinetics and ultimately its bioavailability and efficacy in patients.

### SUMMARY OF THE INVENTION

Described are solid forms of mesembrine (e.g., (−) mesembrine). In some embodiments, solid forms of mesembrine comprise the product of the processes disclosed herein. For example, in some embodiments, mesembrine compositions can be obtained by a process comprising the steps of (a) forming a solution of mesembrine in a solvent, (b) combining the solution from step (a) with a coformer, and (c) obtaining a solid form from the composition of step (b).

In some embodiments, the solid form is crystalline (e.g., a crystalline salt).

In some embodiments, the solid form comprises a coformer.

In some embodiments, the coformer is selected from the coformers in Table 4.

In some embodiments, a solid form salt or solvate of mesembrine (e.g., (−) mesembrine) is obtained by a process comprising the steps described in example 4.

In some embodiments, a solid form salt or solvate is prepared according to example 4.

In some embodiments, a solid form is one of the solid forms described in example 4.

In some embodiments, a solid form salt or solvate of mesembrine (e.g., (−) mesembrine) is obtained by a process comprising the steps described in example 5.

In some embodiments, a solid form salt or solvate is prepared according to example 5.

In some embodiments, a solid form is one of the solid forms described in example 5.

In some embodiments, a solid form salt or solvate of mesembrine (e.g., (−) mesembrine) is obtained by a process comprising the steps described in example 6.

In some embodiments, a solid form salt or solvate is prepared according to example 6.

In some embodiments, a solid form is one of the solid forms described in example 6.

In some embodiments, a solid form salt or solvate of mesembrine (e.g., (−) mesembrine) is obtained by a process comprising the steps described in example 7.

In some embodiments, a solid form salt or solvate is prepared according to example 7.

In some embodiments, a solid form is one of the solid forms described in example 7.

In some embodiments, a solid form salt or solvate of mesembrine (e.g., (−) mesembrine) is obtained by a process comprising the steps described in example 8.

In some embodiments, a solid form salt or solvate is prepared according to example 8.

In some embodiments, a solid form is one of the solid forms described in example 8.

In some embodiments, a solid form salt or solvate of mesembrine (e.g., (−) mesembrine) is obtained by a process comprising the steps described in example 9.

In some embodiments, a solid form salt or solvate is prepared according to example 9.

In some embodiments, a solid form is one of the solid forms described in example 9.

In some embodiments, a solid form salt or solvate of mesembrine (e.g., (−) mesembrine) is obtained by a process comprising the steps described in example 10.

In some embodiments, a solid form salt or solvate is prepared according to example 10.

In some embodiments, a solid form is one of the solid forms described in example 10.

In some embodiments, a solid form salt or solvate of mesembrine (e.g., (−) mesembrine) is obtained by a process comprising the steps described in example 11.

In some embodiments, a solid form salt or solvate is prepared according to example 11.

In some embodiments, a solid form is one of the solid forms described in example 11.

In some embodiments, a pharmaceutical composition comprises a solid form described herein; and a pharmaceutically acceptable excipient.

In some embodiments, a pharmaceutical composition is formed by a process comprising dissolving a solid form described herein.

In some embodiments, a method of treating a mental health disorder, comprises administering to a mammal in need thereof an effective amount of a solid form described herein or a pharmaceutical composition described herein. In some embodiments, the mental health disorder is anxiety, stress, or depression. In some embodiments, the mammal is a human.

### BRIEF DESCRIPTION OF THE DRAWINGS

FIG. 1 XRPD overlay of screening samples with 1 equiv. of glucose by slurry equilibration-RC2;

FIG. 2 XRPD overlay of screening samples with 1 equiv. of Lactose by slurry equilibration-RC4;

FIG. 3 ¹H-NMR spectrum of screening samples with 1 equiv. of Lactose by slurry equilibration-RC4;

FIG. 4 ¹H-NMR spectrum of Lactose;

FIG. 5 XRPD overlay of screening samples with 1 equiv. of L-lysine by slurry equilibration-RC5;

FIG. 6 XRPD overlay of screening samples with 1 equiv. of L-arginine by slurry equilibration-RC6;

FIG. 7 XRPD overlay of screening samples with 1 equiv. of L-phenylalanine by slurry equilibration-RC7;

FIG. 8 XRPD overlay of screening samples with 1 equiv. of meglumine by slurry equilibration-RC9;

FIG. 9 XRPD overlay of screening samples with 1 equiv. of glycine by slurry equilibration-RC10;

FIG. 10 XRPD overlay of screening samples with 1 equiv. of nicotinamide by slurry equilibration-RC11;

FIG. 11 XRPD overlay of screening samples with 1 equiv. of isonicotinamide by slurry equilibration-RC12;

FIG. 12 ¹H-NMR spectrum of screening samples with 1 equiv. of isonicotinamide by slurry equilibration-RC12;

FIG. 13 ¹H-NMR spectrum of isonicotinamide;

FIG. 14 XRPD overlay of screening samples with 1 equiv. of L-proline by slurry equilibration-RC13;

FIG. 15 XRPD overlay of screening samples with 1 equiv. of D-mannitol by slurry equilibration-RC14;

FIG. 16 XRPD overlay of screening samples with 1 equiv. of valerenic acid by slurry equilibration-RC17;

FIG. 17 XRPD overlay of screening samples with 1 equiv. of betulinic acid by slurry equilibration-RC18;

FIG. 18 XRPD overlay of screening samples with 1 equiv. of pamoic acid by slurry equilibration-RC19;

FIG. 19 XRPD overlay of screening samples with 1 equiv. of pamoic acid by slurry equilibration-RC29-1;

FIG. 20 ¹H-NMR spectrum of screening samples with 1 equiv. of pamoic acid by slurry equilibration in ACN/pyridine (90:10, v/v)-RC29;

FIG. 21 XRPD overlay of screening samples with 1 equiv. of pamoic acid by slurry equilibration-RC29-2;

FIG. 22 DSC thermogram of screening samples with 1 equiv. of pamoic acid by slurry equilibration-RC19;

FIG. 23 ¹H-NMR spectrum of screening samples with 1 equiv. of Pamoic acid by slurry equilibration-RC19;

FIG. 24 ¹H-NMR spectrum of batch 2;

FIG. 25 ¹H-NMR spectrum of Pamoic acid;

FIG. 26 ¹H-NMR spectrum of screening samples with 0.5 equiv. of Pamoic acid by slurry equilibration-RC27;

FIG. 27 XRPD overlay of screening samples with 1 equiv. of palmitic acid by slurry equilibration-RC21;

FIG. 28 ¹H-NMR spectrum of screening samples with 1 equiv. of palmitic acid by slurry equilibration-RC21;

FIG. 29 ¹H-NMR spectrum of palmitic acid;

FIG. 30 XRPD overlay of screening samples with 1 equiv. of nicotinic acid by slurry equilibration-RC22;

FIG. 31 XRPD overlay of screening samples with 1 equiv. of folic acid by slurry equilibration-RC24;

FIG. 32 XRPD overlay of screening samples with 1 equiv. of biotin by slurry equilibration-RC25;

FIG. 33 XRPD overlay of screening samples with 1 equiv. of isonicotinamide by slow evaporation-SE12; and

FIG. 34 XRPD overlay of screening samples by salt metathesis.

### DETAILED DESCRIPTION OF THE INVENTION

Applicants have discovered novel solid forms of mesembrine (e.g., (−) mesembrine). Although (−) mesembrine is bioactive with certain desirable pharmacologic effects, certain other properties are less than ideal for use as a therapeutic. Solid forms of mesembrine (e.g., crystalline salts of mesembrine) are described herein.

In some embodiments, solid forms of mesembrine comprise the product of the processes disclosed herein. For example, in some embodiments, mesembrine compositions can be obtained by a process comprising the steps of (a) forming a solution of mesembrine in a solvent, (b) combining the solution from step (a) with a coformer, and (c) obtaining a solid form from the composition of step (b). In some embodiments, solid forms of mesembrine comprise the product of the processes disclosed herein. For example, in some embodiments, mesembrine compositions can be obtained by a process comprising the step of (a) forming a solution of mesembrine in a solvent selected from the group consisting of an alcohol such as methanol, acetone/water or acetonitrile. In some embodiments, mesembrine compositions can be obtained by a process comprising the step of (b) combining the solution from step (a) with a coformer selected from the coformers in Table 2. In some embodiments, mesembrine compositions can be obtained by a process comprising the step of (c) obtaining a solid form from the composition of step (b) by a process selected from the group consisting of slurry equilibration, cooling, temperature cycle and slow evaporation.

Mesembrine can occur in solid forms as an amorphous solid form or in a crystalline solid form or in mixtures of solid forms. Crystalline solid forms of mesembrine can exist in one or more unique solid forms, which can additionally comprise one or more equivalents of water or solvent (i.e., hydrates or solvates, respectively).

Crystalline form(s) of mesembrine having distinct characteristic XRPD peaks are provided herein. Accordingly, provided herein are crystalline mesembrine solid forms, pharmaceutical compositions thereof, and methods of preparing those crystalline mesembrine solid forms and methods of use thereof.

In some embodiments, the solid form comprises mesembrine. In some embodiments, the solid form is crystalline. In some embodiments, the solid form comprises a coformer. In some embodiments, the coformer is selected from the coformers in Table 4.

In some embodiments, a solid form salt or solvate of mesembrine (e.g., (−) mesembrine) is obtained by a process comprising the steps described in example 4. In some embodiments, a solid form salt or solvate is prepared according to example 4. In some embodiments, a solid form is one of the solid forms described in example 4.

In some embodiments, a solid form salt or solvate of mesembrine (e.g., (−) mesembrine) is obtained by a process comprising the steps described in example 5. In some embodiments, a solid form salt or solvate is prepared according to example 5. In some embodiments, a solid form is one of the solid forms described in example 5.

In some embodiments, a solid form salt or solvate of mesembrine (e.g., (−) mesembrine) is obtained by a process comprising the steps described in example 6. In some embodiments, a solid form salt or solvate is prepared according to example 6. In some embodiments, a solid form is one of the solid forms described in example 6.

In some embodiments, a solid form salt or solvate of mesembrine (e.g., (−) mesembrine) is obtained by a process comprising the steps described in example 7. In some embodiments, a solid form salt or solvate is prepared according to example 7. In some embodiments, a solid form is one of the solid forms described in example 7.

In some embodiments, a solid form salt or solvate of mesembrine (e.g., (−) mesembrine) is obtained by a process comprising the steps described in example 8. In some embodiments, a solid form salt or solvate is prepared according to example 8. In some embodiments, a solid form is one of the solid forms described in example 8.

In some embodiments, a solid form salt or solvate of mesembrine (e.g., (−) mesembrine) is obtained by a process comprising the steps described in example 9. In some embodiments, a solid form salt or solvate is prepared according to example 9. In some embodiments, a solid form is one of the solid forms described in example 9.

In some embodiments, a solid form salt or solvate of mesembrine (e.g., (−) mesembrine) is obtained by a process comprising the steps described in example 10. In some embodiments, a solid form salt or solvate is prepared according to example 10. In some embodiments, a solid form is one of the solid forms described in example 10.

In some embodiments, a solid form salt or solvate of mesembrine (e.g., (−) mesembrine) is obtained by a process comprising the steps described in example 11. In some embodiments, a solid form salt or solvate is prepared according to example 11. In some embodiments, a solid form is one of the solid forms described in example 11.

In some embodiments, mesembrine solid form compositions comprise a product of any one of the processes disclosed herein. For example, in some embodiments, mesembrine compositions can be obtained by a process comprising the steps of (a) forming a solution of mesembrine in a solvent comprising methanol or acetonitrile (ACN), (b) combining the solution from step (a) with a conformer (e.g., 1 equiv. conformer), and (c) obtaining a solid form from the composition of step (b). In some embodiments, step (c) can comprise the step of adding L-phenylalanine to the solution of step (b). In some embodiments, step (c) can comprise the step of adding valerenic acid or betulinic acid to the solution of step (b). In some embodiments, step (c) can comprise the step of adding acetone/water (e.g., 9:1, v/v) to the solution of step (b). In some embodiments, step (c) can comprise the step of adding ACN/water (e.g., 95:5, v/v) to the solution of step (b). In some embodiments, step (c) can comprise the step of adding pamoic acid to the solution of step (b). In some embodiments, step (c) can comprise the step of adding acetone/water (e.g., 9:1, v/v) to the solution of step (b). In some embodiments, step (c) can comprise the step of adding pamoic acid were added into 0.54 mL ACN/pyridine (e.g., 90:10, v/v) or 0.5 mL ACN/water/pyridine (e.g., 90:5:5, v/v) to the solution of step (b). In some embodiments, the mesembrine composition is obtained by a process further comprising step (d) of isolating the solid mesembrine composition from the solution of step (b) or step (c).

In some embodiments, the coformer in step (b) is an amino acid. In some embodiments, the conformer in step (b) is selected from glucose, choline, lactose, L-lysine, L-arginine, L-phenylalanine, urea, N-methyl-D-glucamine, glycine, nicotinamide, isonicotinamide, L-proline, D-mannitol, aminobenzoic acid, saccharin, valarenic acid, or betulinic acid. In some embodiments, the conformer in step (b) is selected from pamoic acid, naphthalene-2-sulfonic acid, palmitic acid, nicotinic acid, Vitamin C, folic acid, or biotin.

In some embodiments, the coformer is added as a 0.5-1.0 equiv. amount in step (b). In some embodiments, the coformer is added as 0.5, 0.8 or 1.0 equiv. amount in step (b). In some embodiments, the coformer is added as 1.0 equiv. amount in step (b).

In some embodiments, mesembrine solid form compositions comprise (−) mesembrine and one or more compounds selected from glucose, choline, lactose, L-lysine, L-arginine, L-phenylalanine, urea, N-methyl-D-glucamine, glycine, nicotinamide, isonicotinamide, L-proline, D-mannitol, aminobenzoic acid, saccharin, valarenic acid, and betulinic acid. In some embodiments, mesembrine solid form compositions comprise (−) mesembrine and sodium lauryl sulfonate.

In some embodiments, mesembrine solid form compositions comprise (−) mesembrine and a sugar. In some embodiments, mesembrine solid form compositions comprise (−) mesembrine and glucose, lactose, or D-mannitol. In some embodiments, mesembrine solid form compositions comprise (−) mesembrine and a choline. In some embodiments, mesembrine solid form compositions comprise (−) mesembrine and an amino acid. In some embodiments, mesembrine solid form compositions comprise (−) mesembrine and a glucose derivative. In some embodiments, mesembrine solid form compositions comprise (−) mesembrine and N-methyl-D-glucamine. In some embodiments, mesembrine solid form compositions comprise (−) mesembrine and niacin or a niacin derivative (e.g., nicotinamide or isonicotinamide). In some embodiments, mesembrine solid form compositions comprise (−) mesembrine and an aminobenzoic acid (e.g., PABA). In some embodiments, mesembrine solid form compositions comprise (−) mesembrine and saccharin. In some embodiments, mesembrine solid form compositions comprise (−) mesembrine and L-lysine, L-arginine, L-phenylalanine, glycine, or L-proline. In some embodiments, mesembrine solid form compositions comprise (−) mesembrine and valarenic acid. In some embodiments, mesembrine solid form compositions comprise (−) mesembrine and betulinic acid.

In some embodiments, a pharmaceutical composition comprises a solid form described herein; and a pharmaceutically acceptable excipient. In some embodiments, a pharmaceutical composition is formed by a process comprising dissolving a solid form described herein.

In some embodiments, a method of treating a mental health disorder, comprises administering to a mammal in need thereof an effective amount of a solid form described herein or a pharmaceutical composition described herein. In some embodiments, the mental health disorder is anxiety, stress, or depression. In some embodiments, the mammal is a human

### Pharmaceutical Compositions & Methods of Treatment

In some embodiments, pharmaceutical compositions comprising mesembrine, and pharmaceutically acceptable salts and hydrates thereof are provided. The pharmaceutical composition can comprise mesembrine in one or more solid forms provided herein, such as crystalline mesembrine in a hydrated or anhydrous solid form. A pharmaceutical composition, as used herein, refers to a mixture of mesembrine optionally further comprising other pharmaceutically acceptable components, such as carriers, stabilizers, diluents, dispersing agents, suspending agents, thickening agents, and/or excipients. The pharmaceutical composition can facilitate administration of the compound to a mammal, including compositions formulated for oral administration of mesembrine to a mammal (e.g., capsules or tablets).

In some embodiments, crystalline mesembrine is incorporated into pharmaceutical compositions to provide solid oral dosage forms. In other embodiments, crystalline mesembrine is used to prepare pharmaceutical compositions prepared for oral solid dosage forms. In some embodiments, the pharmaceutical composition comprises an active pharmaceutical ingredient (API) comprising, consisting essentially of, or consisting of mesembrine prepared under applicable Good Manufacturing Practice (GMP). For example, the pharmaceutical composition can be a batch composition comprising mesembrine, wherein the batch composition adheres to Good Manufacturing Practices (e.g., ICH Harmonised Tripartite Guideline, Good Manufacturing Practice Guide for Active Pharmaceutical Ingredients Q7, Current Step 4 version dated 10 Nov. 2010). More preferably, the GMP batch composition can be a homogenous blended batch comprising mesembrine. The FDA (Food and Drug Administration) provides applicable guidance on Good Manufacturing Practice (GMP) for the manufacturing of active pharmaceutical ingredients (APIs) under an appropriate system for managing quality. As used with respect to manufacture of API under GMP, “manufacturing” is defined to include all operations of receipt of materials, production, packaging, repackaging, labelling, relabeling, quality control, release, storage and distribution of APIs and the related controls. An “API Starting Material” is a raw material, intermediate, or an API that is used in the production of an API and that is incorporated as a significant structural fragment into the structure of the API. An API Starting Material can be an article of commerce, a material purchased from one or more suppliers under contract or commercial agreement, or produced in-house. API Starting Materials normally have defined chemical properties and structure.

The pharmaceutical compositions comprising mesembrine can be administered to patients in need thereof, to provide a therapeutically effective amount of a compound of mesembrine.

In practicing the methods of treatment or use provided herein, therapeutically effective amounts of mesembrine are administered in a pharmaceutical composition to a mammal having a disease, disorder, or condition to be treated. In some embodiments, the disease, disorder, or condition is a central nervous system disorder or an inflammatory condition. In some embodiments, pharmaceutical compositions reported herein can be provided in a unit dosage form container (e.g., in a vial or bag or the like).

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, the compound disclosed herein is administered to the patient in a unit dose. In some embodiments, a method comprises the administration to a patient in need thereof of a therapeutically effective amount of a mesembrine composition for the treatment of a disease selected from the group consisting of mild to moderate depression and major depressive episodes. In some embodiments, a method comprises the administration to a patient in need thereof of a therapeutically effective amount of a mesembrine composition for the treatment of anxiety. In some embodiments, a method comprises the administration to a patient in need thereof of a therapeutically effective amount of a mesembrine composition for the treatment of depression. In some embodiments, a method comprises the administration to a patient in need thereof of a therapeutically effective amount of a mesembrine composition for the treatment of a condition selected from the group consisting of: anxiety associated with depression, anxiety with depression, mixed anxiety and depressive disorder. In some embodiments, a method comprises the administration to a patient in need thereof of a therapeutically effective amount of a mesembrine composition for the treatment of anxiety and hysteria or anxiety and depression.

In some embodiments, pharmaceutical compositions reported herein can be provided in an oral dosage form. In some embodiments, an oral dosage form of mesembrine can be a capsule. In some embodiments, an oral dosage form of mesembrine is a tablet. In some embodiments, an oral dosage form comprises a filler. In some embodiments, an oral dosage form comprises two fillers. In some embodiments, an oral dosage form comprises one or more fillers. In some embodiments, an oral dosage form comprises one or more disintegrants. In some embodiments, the oral dosage form comprises one or more lubricants. In some embodiments, an oral dosage form comprises one or more glidants, anti-adherents and/or anti-statics. In some embodiments, an oral dosage form is prepared via dry blending. In some embodiments, an oral dosage form is a tablet and is prepared via dry granulation.

### EXAMPLES

Unless otherwise indicated, the following abbreviations as used herein are defined as indicated in the charts below.

Solvents and Media

| Name | Abbreviation |
|---|---|
| 2-Propanol | IPA |
| Acetonitrile | ACN |
| Dichloromethane | DCM |
| Dimethyl sulfoxide | DMSO |
| Ethanol | EtOH |
| Ethyl acetate | EtOAc |
| Fasted-state simulated intestinal fluid | FaSSIF |
| Fed-state simulated intestinal fluid | FeSSIF |
| Fasted-state simulated gastric fluid | FaSSGF |
| Isopropyl acetate | IPOAc |
| Methanol | MeOH |
| Methyl ethyl ketone | MEK |
| Methyl isobutyl ketone | MIBK |
| tert-Butyl methyl ether | MtBE |
| Tetrahydrofuran | THF |
| Trifluoroacetic acid | TFA |

Units

| Name | Abbreviation |
|---|---|
| Celsius | C. |
| Degree | ° |
| Equivalent(s) | eq. |
| Gram | g |
| Hour | h |
| Joule | J |
| Kelvin | K |
| Liter | L |
| Milligram | mg |
| Milliliter | mL |
| Minute | min |
| Second | s |
| Volume | vol. |
| Weight | wt. |

| Name | Abbreviation |
|---|---|
| Differential Scanning Calorimetry | DSC |
| Dynamic Vapor Sorption | DVS |
| High Performance Liquid Chromatography | HPLC |
| Karl Fischer Titration | KF |
| Nuclear Magnetic Resonance | NMR |
| X-ray Powder Diffraction | XRPD |
| Thermogravimetric Analysis | TGA |

### Example 1—Starting Material Used for Cocrystal Screening

TABLE 1
Starting material

| Compound ID | (−) mesembrine | (−) mesembrine | (−) mesembrine |
|---|---|---|---|
| Batch no. | 1 | 2 | 3 |
| Received | 1.79 g (Oily | 1.56 g (Oily | 2 g (Oily |
| sample size | substance) | substance) | substance) |

TABLE 2
Properties of the starting material as received

| Parameter | Method | Result | Result | Result |
|---|---|---|---|---|
| Batch | N/A | 1 | 2 | 3 |
| Chemical purity | HPLC | >99% | >99% | N/A |
| Chiral purity | HPLC | >99% | >99% | N/A |
| X-ray diffraction | XRPD, 3-40° | N/A | N/A | N/A |
| | (2 theta) | | | |
| Melting onset and | DSC, 10° C./min | N/A | N/A | N/A |
| enthalpy | | | | |
| Thermogravimetry | TGA, 10° C./min | N/A | N/A | N/A |
| Residual solvent(s) | ¹H-NMR (DMSO-d₆) | N/A | N/A | N/A |

### Example 2 Solubility Screening

About 5 mg of batch 1 was weighed into a 2 mL glass vial and aliquots of 20 μL of each solvent were added to get a clear solution. Max. volume of each solvent added was 1 mL. Approximate solubility was determined by visual observation at 25° C.

Based on the solubility results and previous experience, methanol, acetone/water (9:1, v/v) and acetonitrile were selected as screening solvents.

TABLE 1
Approximate solubility at 25° C.

| | | Solubility |
|---|---|---|
| Exp. ID | Solvent | (mg/mL, 25° C.) |
| SL1 | Water | 125-250 |
| SL2 | Methanol | 125-250 |
| SL3 | Ethanol | 125-250 |
| SL4 | Acetone | 125-250 |
| SL5 | Ethyl acetate | 125-250 |
| SL6 | Acetonitrile | 125-250 |
| SL7 | Dichloromethane | 125-250 |
| SL8 | Tetrahydrofuran | 125-250 |
| SL9 | t-Butyl methyl ether | 125-250 |
| SL10 | Toluene | 7-10 |

### Example 3 in Silico Ranking of Cocrystal Formers

Coformer selection: 15 coformers were selected to pursue potential cocrystal opportunities (Table 2).

TABLE 2
Coformers used for cocrystal screening

| Nr. | Counter ions | pKa(s) | M.W. |
|---|---|---|---|
| 1 | Glucose | Neutral | 180.16 |
| 2 | Choline | Basic | 121.18 |
| 3 | Lactose (monohydrate) | Neutral | 360.3 |
| 4 | L-lysine | Basic | 146.19 |
| 5 | L-arginine | Basic | 174.20 |
| 6 | L-phenylalanine | Neutral | 165.19 |
| 7 | Urea | Neutral | 60.06 |
| 8 | N-methyl-D-glucamine (Meglumine) | Basic | 195.22 |
| 9 | Glycine | Neutral | 75.07 |
| 10 | Nicotinamide | Neutral | 122.12 |
| 11 | Isonicotinamide | Neutral | 122.12 |
| 12 | L-proline | Neutral | 115.13 |
| 13 | D-mannitol | Neutral | 182.17 |
| 14 | 4-Aminobenzoic acid | Neutral | 137.14 |
| 15 | Saccharin | Neutral | 183.18 |
| 16 | Valerenic acid | Acidic | 234.33 |
| 17 | Betulinic acid | Acidic | 456.7 |
| 18 | Pamoic acid | Acidic | 388.37 |
| 19 | Naphthalene-2-sulfonic acid | Acidic | 208.23 |
| 20 | Palmitic acid | Acidic | 256.42 |
| 21 | Nicotinic acid | Acidic | 123.11 |
| 1 | Glucose | Neutral | 180.16 |
| 2 | Choline | Basic | 121.18 |
| 22 | Vitamin C | Acidic | 176.12 |
| 23 | Folic acid | Acidic | 441.4 |
| 24 | Biotin | Acidic | 244.31 |
| 25 | Sodium laurylsulfonate | Acidic | 272.38 |

### Example 4 Screening Experiments

With the selected coformers and the selected solvents, slurry equilibration, cooling, temperature cycle and slow evaporation were applied as screening methods.

The screening results were summarized in Table 3, Table 4, Table 6, Table 8 and Table 9. All these cocrystal hits will be further characterized.

### Example 5 Slurry Equilibration Experiment-1 (Table 3)

1. 750-800 mg of batch 1 was dissolved into 4.8 ml of methanol or 4.5 mL of ACN. Obtained clear solutions were dispensed into 2 ml vials. 1 equiv. of coformers was added into the glass vial (RC2A-RC6A, RC7A-RC16A, RC2C-RC6C and RC7C-RC16C).
2. About 30 mg of batch 2 and 1 equiv. of L-phenylalanine were added into MeOH or ACN in a 2 mL glass vial, respectively.
3. About 30 mg of batch 2 and 1 equiv. of valerenic acid or betulinic acid were added into MeOH or acetone/water (9:1, v/v) or ACN in a 2 mL glass vial, respectively.
4. About 600 mg of batch 2 was dissolved into 6 ml of acetone/water (9:1, v/v). Obtained clear solutions were dispensed into 2 ml vials. 1 equiv. of coformer was added into the glass vial (RC2B-RC6B and RC7B-RC16B).
   Obtained above mixtures were stirred at 50° C. for 1 hour and then at 25° C. for 4 days.

### Example 6 Slurry Equilibration Experiment-2 (Table 6)

1. About 30 mg of batch 2 and 1 equiv. of coformers were added into water in a 2 mL glass vial, respectively. (RC19D-RC19E)
2. About 210 mg of batch 2 was dissolved into 2.1 ml of ACN/water (95:5, v/v). Obtained clear solutions were dispensed into 2 ml vials. 1 equiv. of coformer was added into the glass vial. (RC19E-RC24E)
3. About 30 mg of batch 2 was dissolved into 0.3 ml of water or ACN/water (95:5, v/v). Obtained solutions were dispensed into 2 ml vials. 1 equiv. of biotin was added into the glass vial. (RC25D and RC25E)
4. About 50 mg of batch 3 was dissolved into 0.3 ml of ACN/water (95:5, v/v). Obtained clear solutions were dispensed into 2 ml vials. 0.5 equiv. of pamoic acid was added into the glass vial. (RC27E)
5. The sample of RC27-E and additional 0.3 equiv. of pamoic acid were slurried in mother liquid of RC27-E, after stirred at 25° C. for about 1 day. Additional 0.3 ml of ACN/water (95:5, v/v) was added into the glass vial. (RC28E)
   Obtained above mixtures were stirred at 50° C. for 1 hour and then at 25° C. for 10-11 days.

### Example 7 Slurry Equilibration Experiment-3 (Table 3)

1. About 50 mg of batch 3 and 1 equiv. of pamoic acid were added into 0.54 mL ACN/pyridine (90:10, v/v) or 0.5 mL ACN/water/pyridine (90:5:5, v/v) in a 2 mL glass vial, respectively.
   Obtained mixtures were stirred at 50° C. for 1 hour and then at 25° C.

Obtained suspensions were filtered through a 0.45 μm nylon membrane filter by centrifugation at 14,000 rpm. Solids were analyzed by XRPD (Table 3, Table 4 and Table 5). Thin suspension samples were used for equilibration under temperature cycle attempting to obtain sufficient solids for characterization. Obtained clear solutions were placed at 5° C. to get precipitate. If still no solids precipitate, then the solutions were treated by slow evaporation at ambient condition.

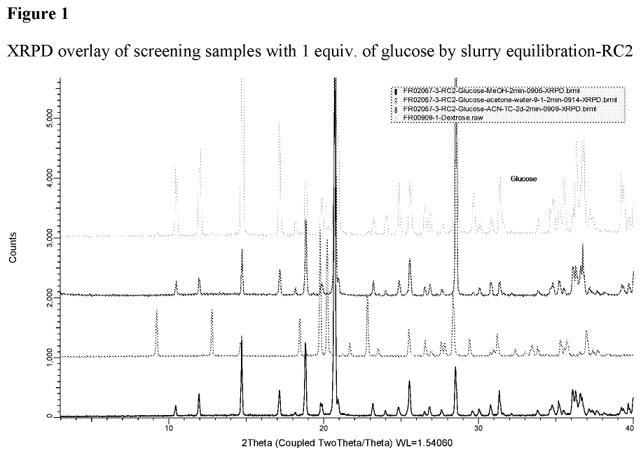

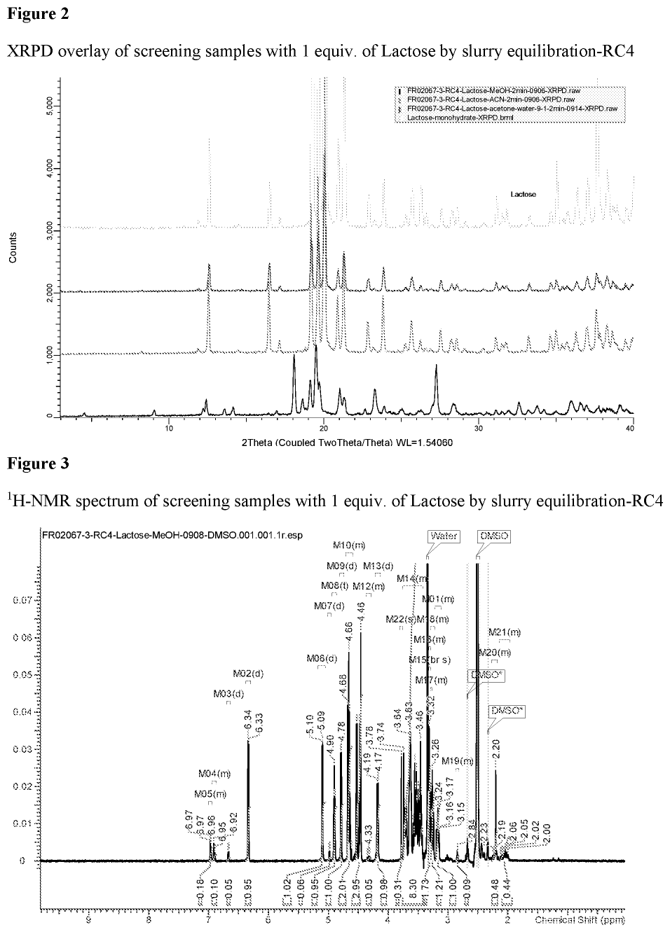

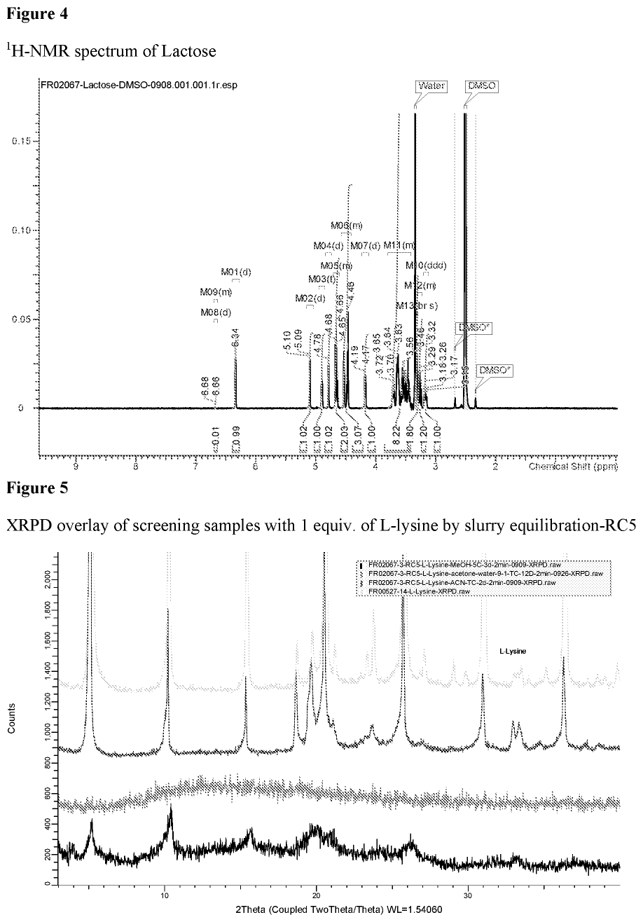

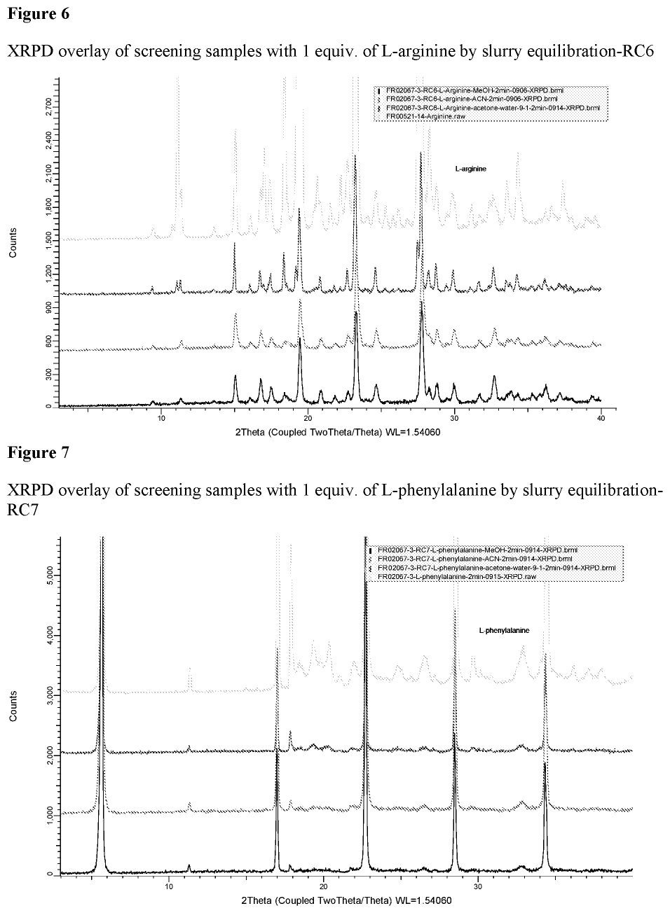

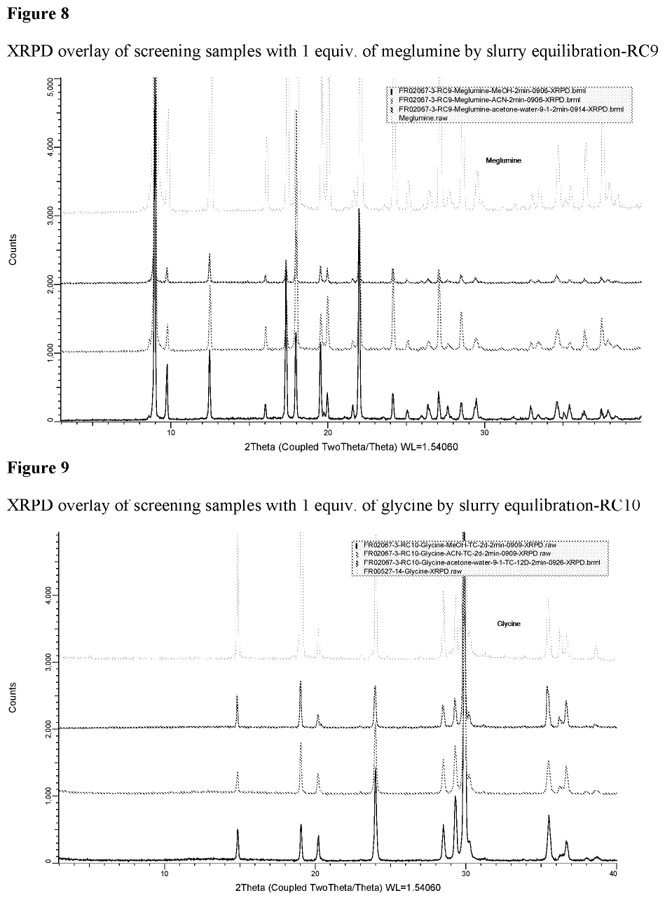

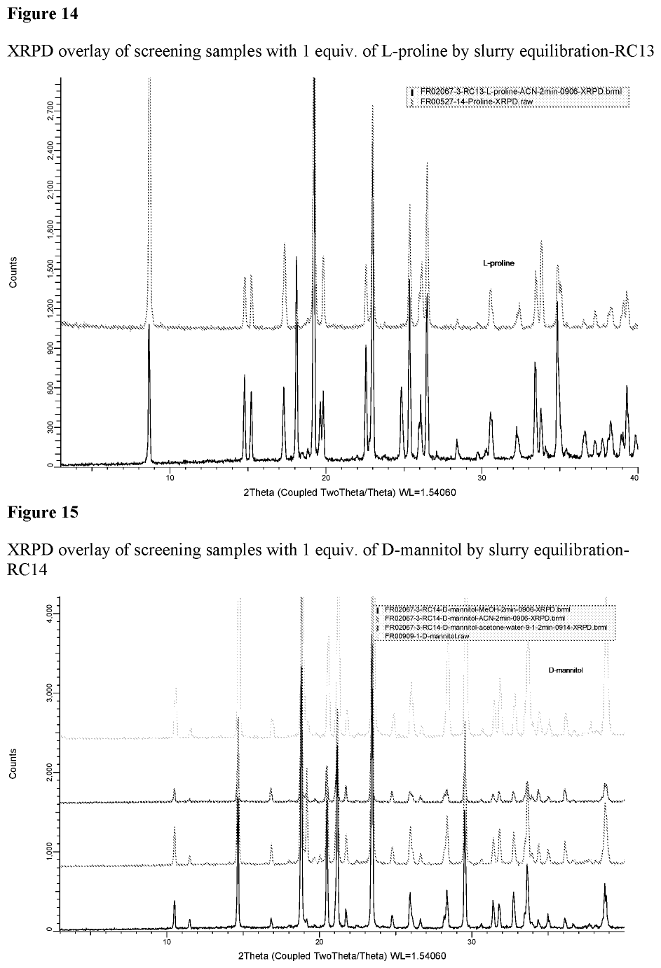

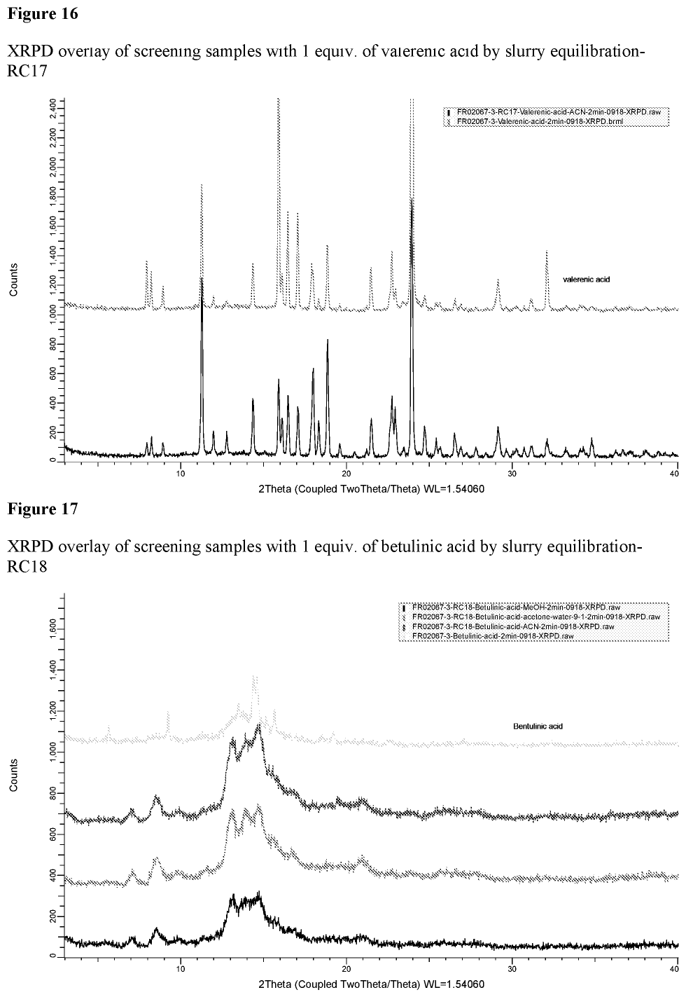

TABLE 3
Slurry equilibration-1

| | | | B | |
|---|---|---|---|---|
| Exp. | | A | Acetone/water | C |
| ID | Coformers | Methanol | (9:1, v/v) | ACN |
| RC1 | Free form | Clear solution | Clear solution | Clear solution |
| | | (yellow) | | (yellow) |
| RC2 | 1.0 equiv. | Glucose FIG. 1 | Glucose FIG. 1 | Thin suspension + |
| | Glucose | | | solids (yellow) → |
| | | | | equilibration at 25° C. |
| | | | | for about 4 days → |
| | | | | suspension + solids |
| | | | | (off-white) |
| RC3 | 1.0 equiv. | Clear solution | Clear solution | Clear solution |
| | Choline | (brown) → | (brown) | (brown) |
| | | equilibration at 25° C. | | |
| | | for about 4 days → | | |
| | | Clear solution | | |
| | | (yellow) | | |
| RC4 | 1.0 equiv. | Lactose | Lactose | Lactose |
| | Lactose | (monohydrate) | (monohydrate) | (monohydrate) |
| | | FIG. 2; | FIG. 2 | FIG. 2 |
| | | ¹HNMR (DMSO- | | |
| | | d₆): lactose FIG. 3 | | |
| RC5 | 1.0 equiv. | Thin suspension | Hazy solution + | Thin suspension + |
| | L-lysine | (yellow) | Brown solids on the | solids on the bottom |
| | | | bottom → | (yellow) |
| | | | equilibration at 25° C. | |
| | | | for about 4 days | |
| | | | →Clear solution + | |
| | | | oil like solids (black) | |
| RC6 | 1.0 equiv. L- | L-arginine FIG. 6 | L-arginine FIG. 6 | L-arginine FIG. 6 |
| | arginine | | | |
| RC7 | 1.0 equiv. L- | L-phenylalanine | L-phenylalanine | L-phenylalanine |
| | phenylalanine | FIG. 7 | FIG. 7 | FIG. 7 |
| RC8 | 1.0 equiv. Urea | Clear solution | Clear solution | Clear solution + |
| | | (yellow) | | solids on the |
| | | | | bottom (yellow) |
| RC9 | 1.0 equiv. N- | Meglumine FIG. 8 | Meglumine FIG. 8 | Meglumine FIG. 8 |
| | methyl-D- | | | |
| | glucamine | | | |
| | (Meglumine) | | | |
| RC10 | 1.0 equiv. | Thin suspension + | Clear solution + few | Thin suspension + |
| | Glycine | solids on the bottom | solids on the bottom | solids on the bottom |
| | | (yellow) | (off-white) → | (yellow) |
| | | | equilibration at 25° C. | |
| | | | for about 4 days → | |
| | | | hazy solution + | |
| | | | solids (off-white) | |
| RC11 | 1.0 equiv. | Clear solution | Clear solution | Thin suspension + |
| | Nicotinamide | (yellow) | | solids on the bottom |
| | | | | (yellow) |
| RC12 | 1.0 equiv. | Clear solution | Clear solution | Clear solution + |
| | Isonicotinamide | (yellow) | | solids on the bottom |
| | | | | (yellow) |
| RC13 | 1.0 equiv. L- | Clear solution | Clear solution | L-proline FIG. 14 |
| | proline | (yellow) | | |
| RC14 | 1.0 equiv. D- | D-mannitol FIG. 15 | D-mannitol FIG. 15 | D-mannitol FIG. 15 |
| | mannitol | | | |
| RC15 | 1.0 equiv. 4- | Clear solution | Clear solution | Clear solution |
| | Aminobenzoic | (yellow) | | (yellow) |
| | acid | | | |
| RC16 | 1.0 equiv. | Clear solution | Clear solution | Clear solution |
| | Saccharin | (yellow) | | (yellow) |
| RC17 | 1.0 equiv. | Clear solution | Clear solution | Valerenic acid |
| | Valerenic acid | | | FIG. 16 |
| RC18 | 1.0 equiv. | Betulinic acid | Betulinic acid | Betulinic acid |
| | Betulinic acid | FIG. 17 | FIG. 17 | FIG. 17 |

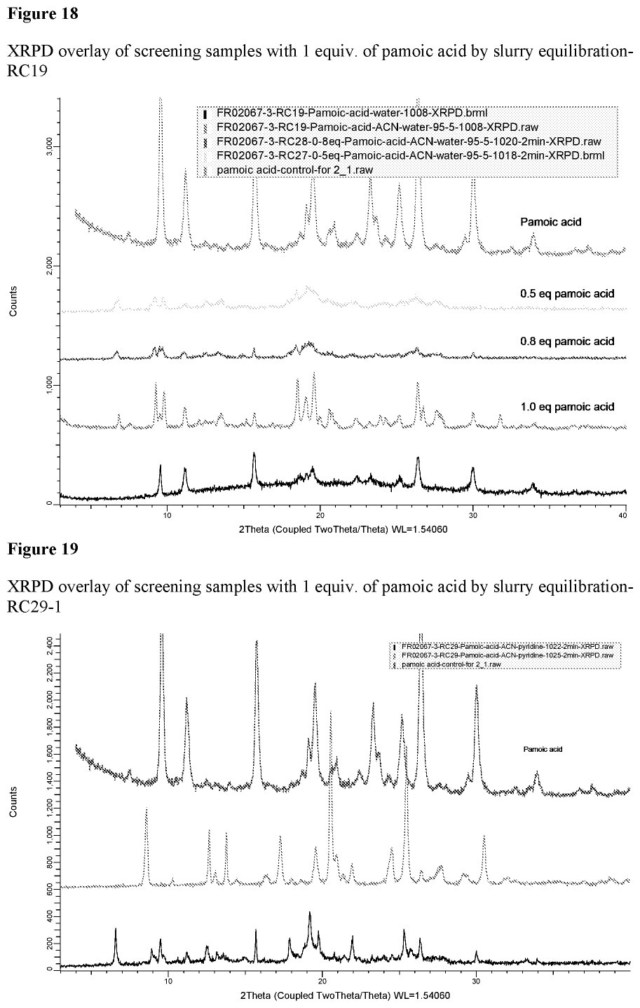

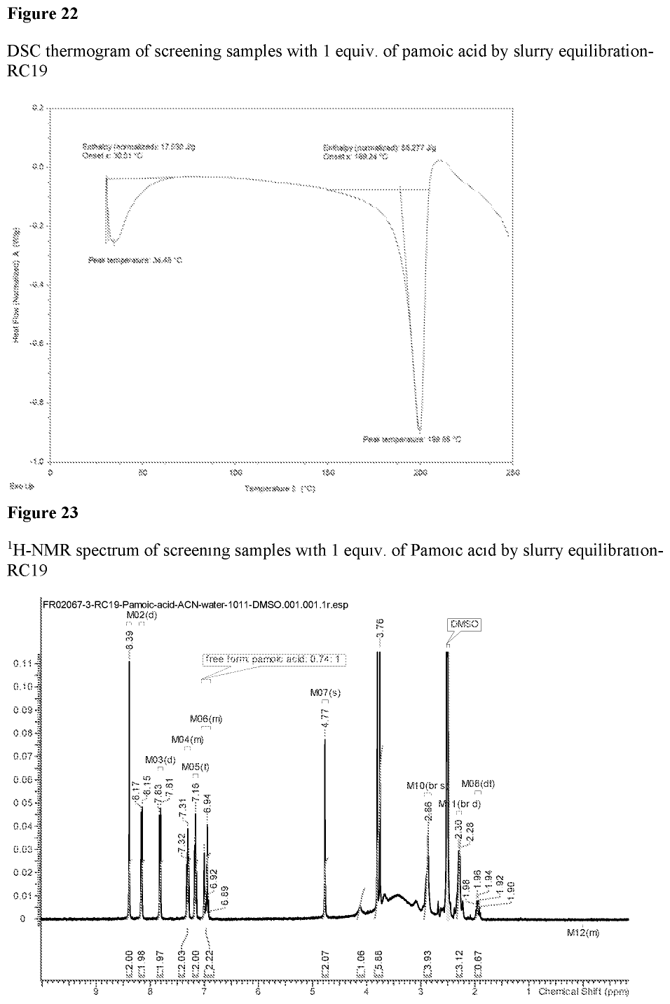

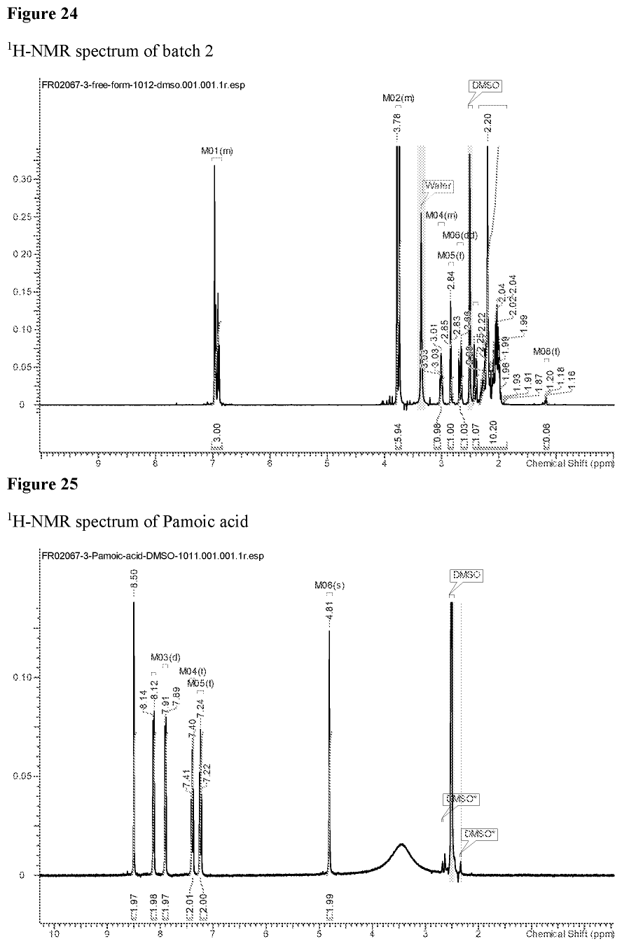

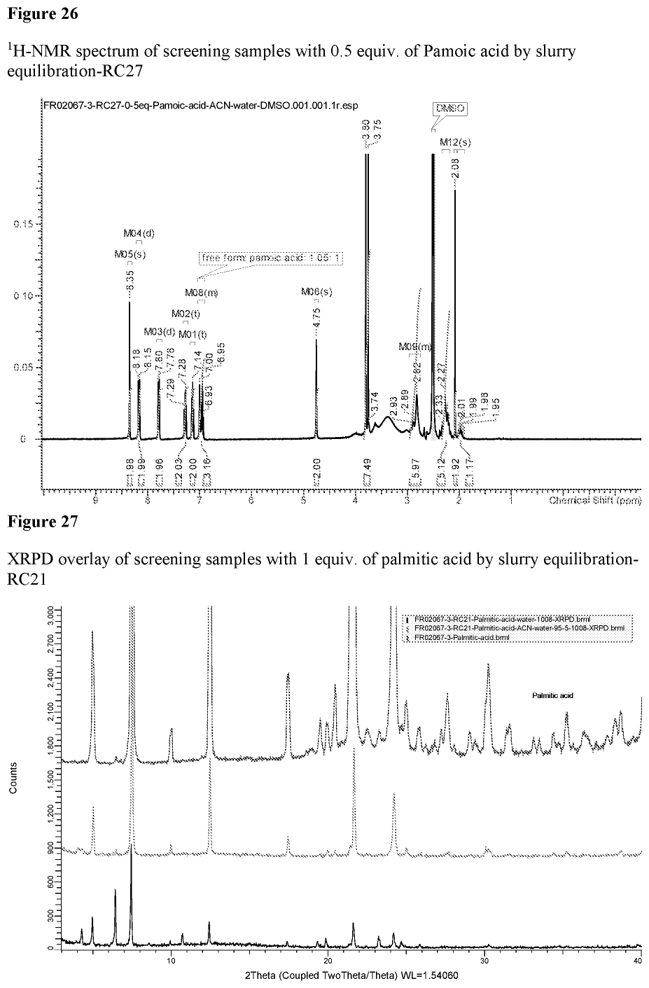

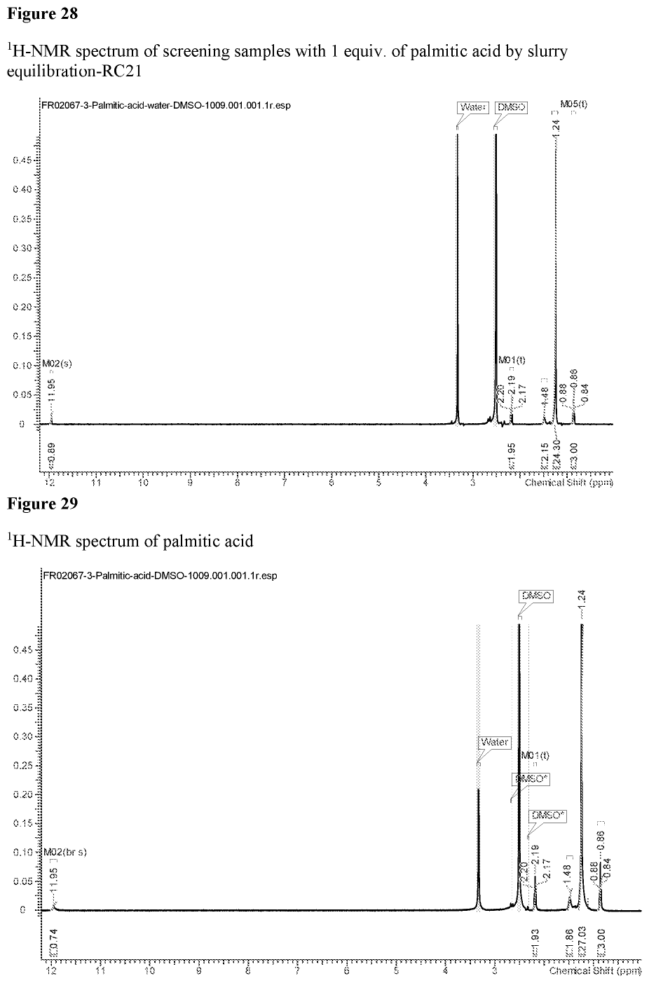

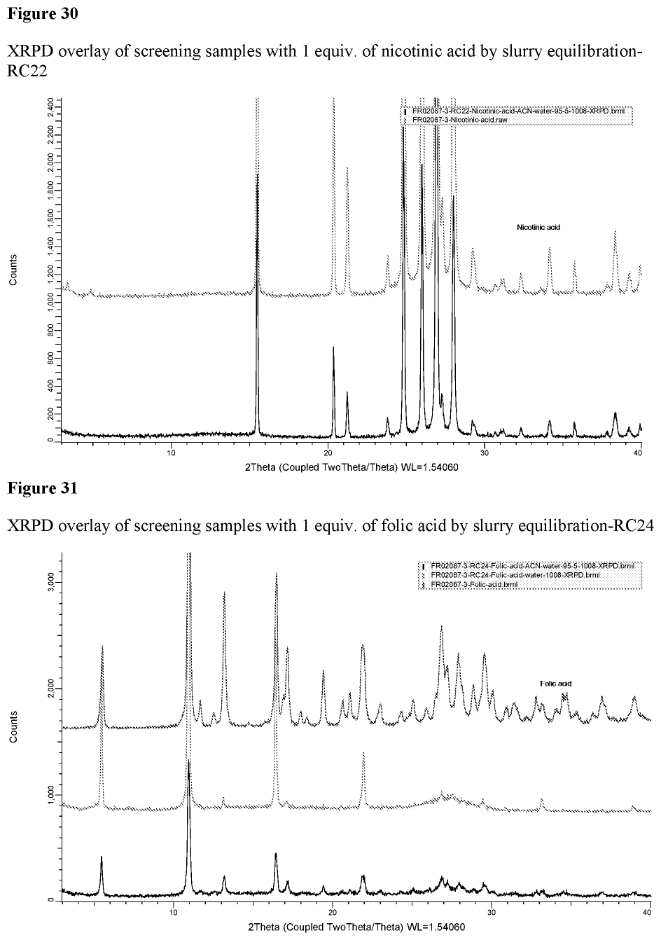

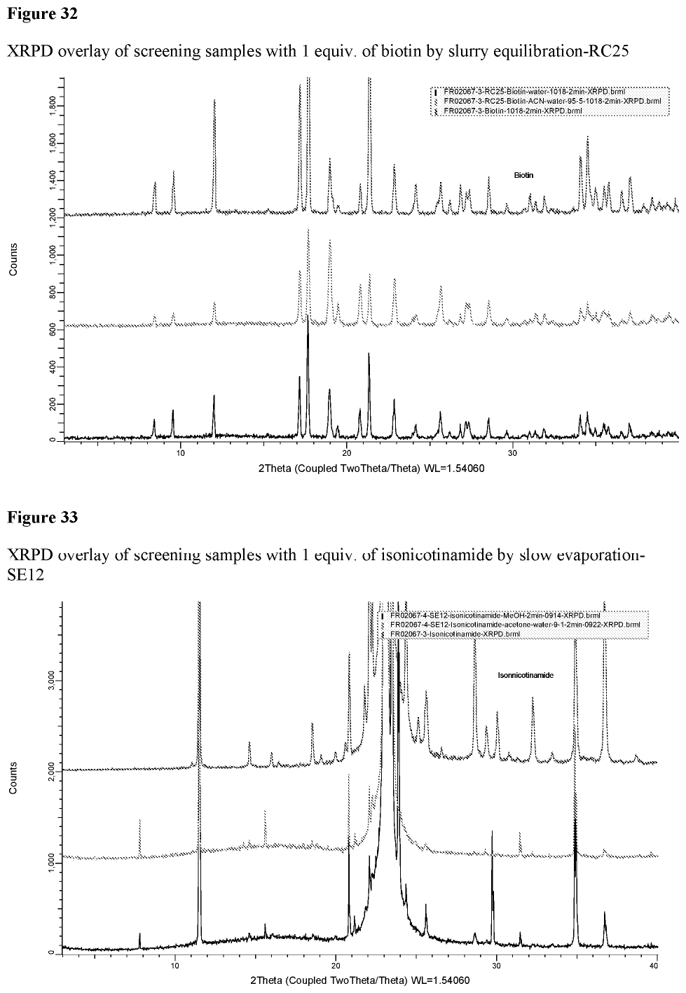

TABLE 4
Slurry equilibration-2

| Exp. | | D | E |
|---|---|---|---|
| ID | Coformers | Water | ACN/water (95:5, v/v) |
| RC19 | 1.0 equiv. | Clear solution + green stick | Suspension → equilibration at |
| | Pamoic acid | solids → equilibration at 25° C. | 25° C. for about 11 days → |
| | | for about 11 days →Clear | Suspension (turquoise); |
| | | solution + green stick solids; | XRPD: Mixture of pamoic acid |
| | | XRPD: Pamoic acid FIG. 18 | and pamoate salt Pattern A |
| | | | FIG. 18; |
| | | | DSC: dehydration onset: |
| | | | 30.5° C.(17 J/g), melting onset: |
| | | | 189.2° C. (58 J/g) FIG. 22; |
| | | | ¹H-NMR (DMSO-d₆): free form: |
| | | | pamoic acid = 0.74: 1 FIG. 23, |
| | | | FIG. 24, FIG. 25 |
| RC20 | 1.0 equiv. | Clear solution → equilibration at | Clear solution → equilibration at |
| | Naphthalene- | 25° C. for about 11 days → Clear | 25° C. for about 11 days → Clear |
| | 2-sulfonic acid | solution | solution |
| RC21 | 1.0 equiv. | Thin suspension (off-white) → | Jelly-like (off-white) → |
| | Palmitic acid | equilibration at 25° C. for about 11 | equilibration at 25° C. for about |
| | | days → Suspension (off-white); | 11 days → Suspension (off- |
| | | XRPD: Palmitic acid + two peaks | white); |
| | | at 2theta 4.2° and 10.7° FIG. 27; | XRPD: Palmitic acid FIG. 27; |
| | | ¹H-NMR (DMSO-d₆): Palmitic | |
| | | FIGS. 28, 29 | |
| RC22 | 1.0 equiv. | Clear solution → equilibration at | Clear solution + solids → |
| | Nicotinic acid | 25° C. for about 11 days → Clear | equilibration at 25° C. for about |
| | | solution | 11 days → Clear solution + |
| | | | solids |
| | | | XRPD: Nicotinic acid FIG. 30 |
| RC23 | 1.0 equiv. | Clear solution (brown) → | Hazy solution (brown) → |
| | Vitamin C | equilibration at 25° C. for | equilibration at 25° C. for about |
| | | about 10 days → Clear solution | 11 days → Clear solution + |
| | | (brown) | brown oil on the bottom (brown) |
| RC24 | 1.0 equiv. | Suspension (orange) → | Suspension (orange) → |
| | Folic acid | equilibration at 25° C. for about | equilibration at 25° C. for about |
| | | 10 days → Solids (orange); | 11 days →Suspension (orange); |
| | | XRPD: Folic acid FIG. 31 | XRPD: Folic acid FIG. 31 |
| RC25 | 1.0 equiv. | Hazy solution + solids → | Suspension → equilibration at |
| | Biotin | equilibration at 25° C. for about 4 | 25° C. for about 4 days → |
| | | days → Hazy solution + solids; | suspension (off-white); |
| | | XRPD: Biotin FIG. 32 | XRPD: Biotin FIG. 32 |
| RC26 | Free form | Hazy solution → equilibration at | Clear solution → equilibration at |
| | | 25° C. for about 10 days → Hazy | 25° C. for about 10 days → Clear |
| | | solution | solution |
| RC27 | 0.5 equiv. | // | Suspension → equilibration at |
| | Pamoic acid | | 25° C. for about 4 days → |
| | | | suspension (off-white); |
| | | | XRPD: Pamoate salt Pattern A |
| | | | (low crystallinity) FIG. 18; |
| | | | ¹H-NMR (DMSO-d₆): free form: |
| | | | pamoic acid = 1.05: 1 FIG. 26 |
| RC28 | 0.8 equiv. | // | Suspension → equilibration at |
| | Pamoic acid | | 25° C. for about 2 days → |
| | | | suspension (yellow); |
| | | | XRPD: Mixture of pamoic acid |
| | | | and pamoate salt Pattern A |
| | | | FIG. 18 |

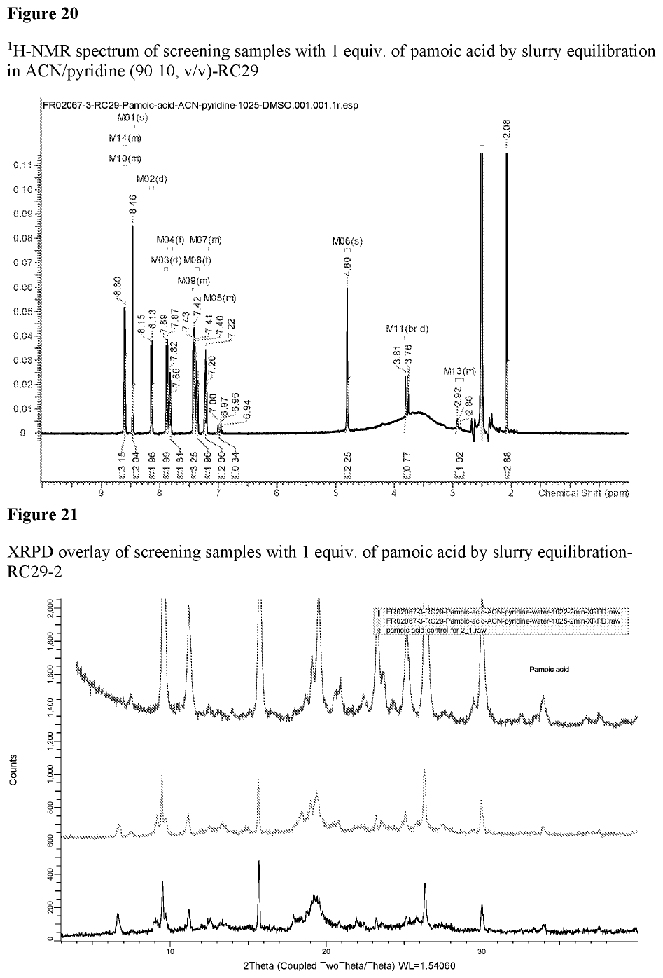

TABLE 5
Slurry equilibration-3

| Exp. | | F | G |
|---|---|---|---|
| ID | Coformers | ACN/pyridine (90:10, v/v) | ACN/pyridine/water (90:5:5, v/v) |
| RC29 | 1.0 equiv. | Suspension (yellow) → | Suspension → equilibration at |
| | Pamoic acid | equilibration at 25° C. for about | 25° C. for about 4 days → |
| | | 4 days → suspension (off-white); | suspension (yellow); |
| | | XRPD: Pyridine solvate of | XRPD: Mixture of pamoic acid |
| | | Pamoic acid FIG. 19; | and pamoate salt Pattern A |
| | | ¹H-NMR (DMSO-d₆): FIG. 20 | FIG. 21 |

### Example 8 Cooling Experiments

The clear solutions obtained from slurry equilibration experiments were further cooled to 5° C. The results were summarized in Table 6 and Table 7.

TABLE 6
Cooling experiments-1

| | | | B | |
|---|---|---|---|---|
| Exp. | | A | Acetone/water | C |
| ID | Coformers | Methanol | (9:1, v/v) | ACN |
| C1 | Free form | Placed at 5° C. for about | Placed at 5° C. for | Placed at 5° C. for |
| | | 3 days → clear | about 5 days → | about 3 days → clear |
| | | solution (yellow) | clear solution | solution, (yellow) |
| | | | (yellow) | |
| C2 | 1.0 equiv. | // | // | // |
| | Glucose | | | |
| C3 | 1.0 equiv. | Placed at 5° C. for about | Placed at 5° C. for | Placed at 5° C. for |
| | Choline | 3 day → clear solution | about 5 days → | about 3 day → clear |
| | | (yellow) | clear solution | solution (brown oil on |
| | | | (brown) | the wall of bottle) |
| C4 | 1.0 equiv. | // | // | // |
| | Lactose | | | |
| C5 | 1.0 equiv. L- | Placed at 5° C. for about | // | // |
| | lysine | 3 days → thin | | |
| | | suspension (yellow); | | |
| | | XRPD: L-lysine (low | | |
| | | crystallinity) FIG. 5 | | |
| C6 | 1.0 equiv. L- | // | // | // |
| | arginine | | | |
| C7 | 1.0 equiv. L- | // | // | // |
| | phenylalanine | | | |
| C8 | 1.0 equiv. | Placed at 5° C. for about | Placed at 5° C. for | // |
| | Urea | 3 day → clear solution | about 5 days → | |
| | | (yellow) | clear solution | |
| | | | (yellow) | |
| C9 | 1.0 equiv. N- | // | // | // |
| | methyl-D- | | | |
| | glucamine | | | |
| | (Meglumine) | | | |
| C10 | 1.0 equiv. | // | // | // |
| | Glycine | | | |
| C11 | 1.0 equiv. | Placed at 5° C. for about | Placed at 5° C. for | // |
| | Nicotinamide | 3 day → clear solution | about 5 days → | |
| | | (yellow) | clear solution | |
| | | | (yellow) | |
| C12 | 1.0 equiv. | Placed at 5° C. for about | Placed at 5° C. for | // |
| | Isonicotinamide | 3 days → clear | about 5 days → | |
| | | solution (yellow) | clear solution | |
| | | | (yellow) | |
| C13 | 1.0 equiv. L- | Placed at 5° C. for about | Placed at 5° C. for | // |
| | proline | 3 days → clear | about 5 days → | |
| | | solution (brown) | clear solution | |
| | | | (yellow) | |
| C14 | 1.0 equiv. D- | // | // | // |
| | mannitol | | | |
| C15 | 1.0 equiv. 4- | Placed at 5° C. for about | Placed at 5° C. for | Placed at 5° C. for |
| | Aminobenzoic | 3 days → clear | about 5 days → | about 3 days → clear |
| | acid | solution (yellow) | clear solution | solution (yellow) |
| | | | (yellow) | |
| C16 | 1.0 equiv. | Placed at 5° C. for about | Placed at 5° C. for | Placed at 5° C. for |
| | Saccharin | 3 days → clear | about 5 days → | about 3 days → clear |
| | | solution (yellow) | clear solution | solution (yellow) |
| | | | (yellow) | |
| C17 | 1.0 equiv. | Placed at 5° C. for 8 | Placed at 5° C. for | // |
| | Valerenic acid | days → clear solution | 8 days → clear | |
| | | | solution | |
| C18 | 1.0 equiv. | // | // | // |
| | Betulinic acid | | | |

TABLE 7
Cooling experiments-2

| Exp. | | D | E |
|---|---|---|---|
| ID | Coformers | Water | ACN/ water (95:5, v/v) |
| C19 | 1.0 equiv. | // | // |
| | Pamoic acid | | |
| C20 | 1.0 equiv. | Placed at 5° C. for | Placed at 5° C. for |
| | Naphthalene- | 4 days → Clear | 4 days → Clear |
| | 2-sulfonic acid | solution | solution |
| C21 | 1.0 equiv. | // | // |
| | Palmitic acid | | |
| C22 | 1.0 equiv. | Placed at 5° C. for | // |
| | Nicotinic acid | 4 days → Clear | |
| | | solution | |
| C23 | 1.0 equiv. | Placed at 5° C. for | Placed at 5° C. for |
| | Vitamin C | 4 days → Clear | 4 days → Clear |
| | | solution | solution + brown oil |
| | | | on the bottom |
| C24 | 1.0 equiv. | // | // |
| | Folic acid | | |
| C25 | 1.0 equiv. | // | // |
| | Biotin | | |
| C26 | Free form | Placed at 5° C. for | Placed at 5° C. for |
| | | 4 days → Hazy | 4 days → Clear |
| | | solution | solution |

Explanation “//”: Not carried out.

### Example 9 Equilibration Under Temperature Cycle

For samples with thin suspension obtained from slurry equilibration, they were further used for equilibration under temperature cycle between 5° C. and 50° C. The results were summarized in Table 8.

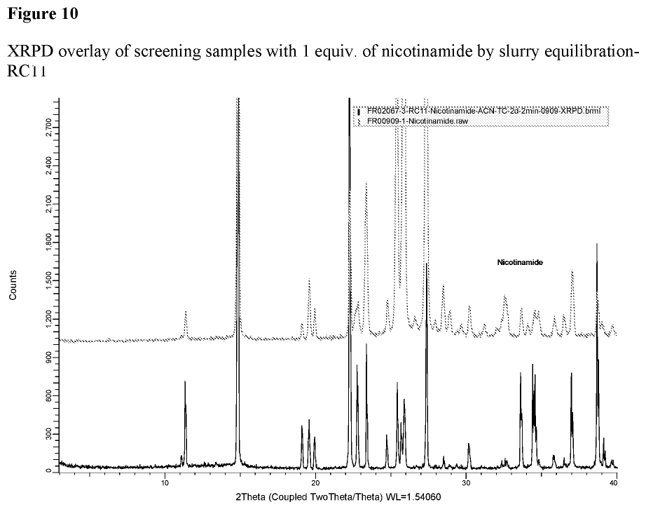

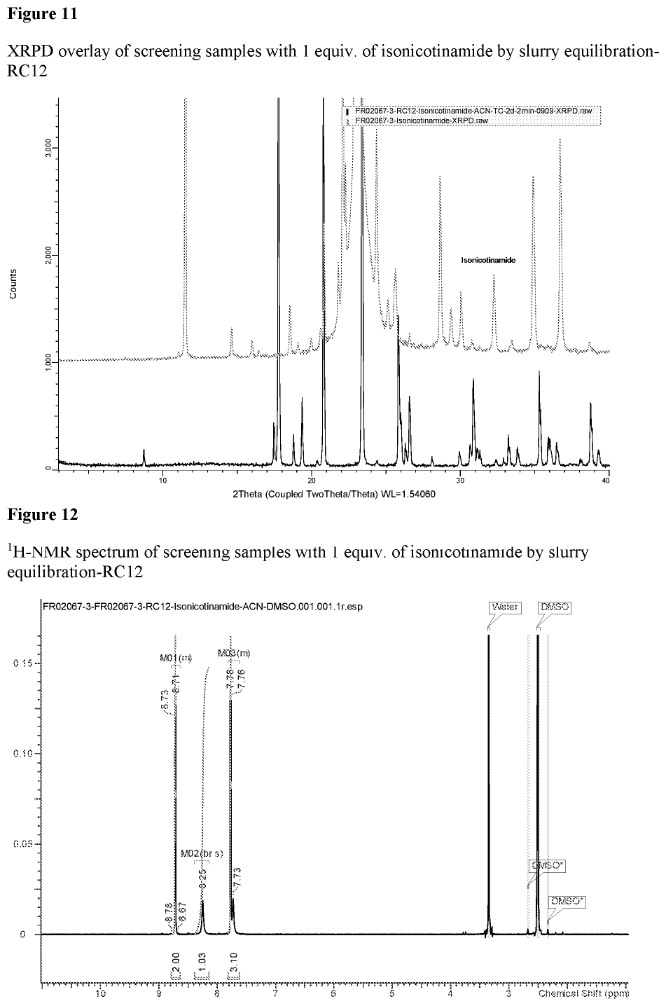

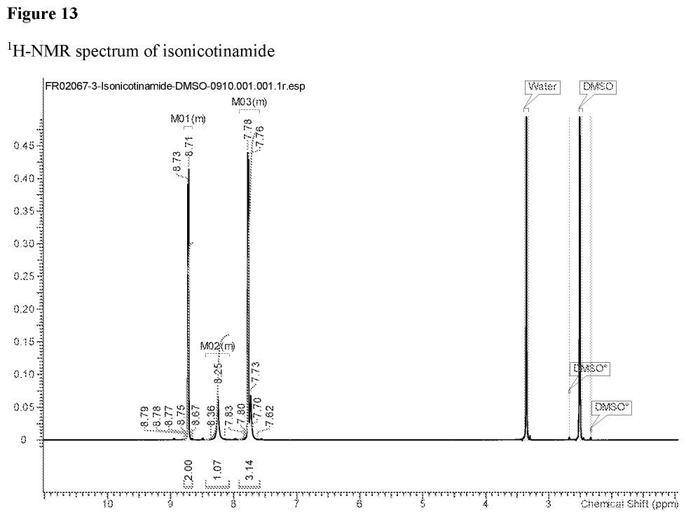

TABLE 8
Equilibration under temperature cycle

| Exp. | | A | B | C |
|---|---|---|---|---|
| ID | Coformers | Methanol | Acetone/water(9:1) | ACN |
| TC1 | Free form | // | // | // |
| TC2 | 1.0 equiv. | // | // | Equilibration under |
| | Glucose | | | temperature cycle for |
| | | | | 2 days → suspension + |
| | | | | solids (off-white) |
| | | | | XRPD: glucose FIG. 1 |
| TC3 | 1.0 equiv. | // | // | // |
| | Choline | | | |
| TC4 | 1.0 equiv. | // | // | // |
| | Lactose | | | |
| TC5 | 1.0 equiv. L- | // | Equilibration under | Equilibration under |
| | lysine | | temperature cycle | temperature cycle for |
| | | | for about 12 days | about 2 days → thin |
| | | | → Clear solution + | suspension + solids |
| | | | oil like solids | (yellow); |
| | | | (black); | XRPD: L-lysine |
| | | | XRPD: Amorphous | |
| | | | FIG. 5 | |
| TC6 | 1.0 equiv. L- | // | // | // |
| | arginine | | | |
| TC7 | 1.0 equiv. L- | // | // | // |
| | phenylalanine | | | |
| TC8 | 1.0 equiv. Urea | // | // | Clear solution +solids |
| | | | | (yellow) → |
| | | | | equilibration under |
| | | | | temperature cycle for |
| | | | | about 2 days → clear |
| | | | | solution + solids |
| | | | | (yellow); |
| TC9 | 1.0 equiv. N- | // | // | // |
| | methyl-D- | | | |
| | glucamine | | | |
| | (Meglumine) | | | |
| TC10 | 1.0 equiv. | Equilibration under | Equilibration under | Equilibration under |
| | Glycine | temperature cycle for | temperature cycle | temperature cycle for |
| | | about 2 days → | for about 12 days | about 2 days |
| | | suspension + few | → Thin suspension + | →suspension + few |
| | | solids (yellow); | solids; XRPD: | solids (yellow); |
| | | XRPD: Glycine FIG. 9 | Glycine FIG. 9 | XRPD: Glycine FIG. 9 |
| TC11 | 1.0 equiv. | // | // | Equilibration under |
| | Nicotinamide | | | temperature cycle for |
| | | | | about 2 days → clear |
| | | | | solution + needlelike |
| | | | | crystal (in low |
| | | | | temperature); XRPD: |
| | | | | nicotinamide FIG. 10 |
| TC12 | 1.0 equiv. | // | // | Equilibration under |
| | Isonicotinamide | | | temperature cycle for |
| | | | | 2 days → suspension + |
| | | | | solids (off-white); |
| | | | | XRPD: isonicotinamide |
| | | | | FIG. 11; ¹H-NMR |
| | | | | (DMSO-d₆): isonicotinamide |
| | | | | FIGS. 12, 13 |
| TC13 | 1.0 equiv. L- | // | // | // |
| | proline | | | |
| TC14 | 1.0 equiv. D- | // | // | // |
| | mannitol | | | |
| TC15 | 1.0 equiv. 4- | // | // | // |
| | Aminobenzoic | | | |
| | acid | | | |
| TC16 | 1.0 equiv. | // | // | // |
| | Saccharin | | | |
| TC17 | 1.0 equiv. | // | // | // |
| | Valerenic acid | | | |
| TC18 | 1.0 equiv. | // | // | // |
| | Betulinic acid | | | |

Explanation “//”: Not carried out

### Example 10 Slow Evaporation

Clear solutions obtained from slow cooling experiments were further treated by slow evaporation under ambient condition.

The results were summarized in Table 9 and

### Table 10.

TABLE 9
Slow evaporation-1

| | | | B | |
|---|---|---|---|---|
| Exp. | | A | Acetone/water | C |
| ID | Coformers | Methanol | (9:1, v/v) | ACN |
| SE1 | Free form | Gel-like | Gel-like | Gel-like |
| SE2 | 1.0 equiv. Glucose | // | // | // |
| SE3 | 1.0 equiv. Choline | Gel-like | Gel-like | Gel-like |
| SE4 | 1.0 equiv. Lactose | // | // | // |
| SE5 | 1.0 equiv. L-lysine | // | // | // |
| SE6 | 1.0 equiv. L-arginine | // | // | // |
| SE7 | 1.0 equiv. L-phenylalanine | // | // | // |
| SE8 | 1.0 equiv. Urea | Gel-like | Gel-like | Add 0.1 mL MeOH → |
| | | | | clear solution; Gel-like |
| SE9 | 1.0 equiv. N-methyl-D-glucamine | // | // | // |
| | (Meglumine) | | | |
| SE10 | 1.0 equiv. Glycine | // | // | // |
| SE11 | 1.0 equiv. Nicotinamide | Gel-like | Gel-like | // |
| SE12 | 1.0 equiv. Isonicotinamide | Rod-like solids; | Rod-like solids; | // |
| | | XRPD: isonicotinamide | XRPD: isonicotinamide | |
| | | FIG. 33 | FIG. 33 | |
| SE13 | 1.0 equiv. L-proline | Gel-like | Gel-like | // |
| SE14 | 1.0 equiv. D-mannitol | // | // | // |
| SE15 | 1.0 equiv. 4-Aminobenzoic acid | Gel-like | Gel-like | Gel-like |
| SE16 | 1.0 equiv. Saccharin | Gel-like | Gel-like | Gel-like |
| SE17 | 1.0 equiv. Valerenic acid | // | // | // |
| SE18 | 1.0 equiv. Betulinic acid | // | // | // |

TABLE 10
Slow evaporation-2

| Exp. | | D | E |
|---|---|---|---|
| ID | Coformers | Water | ACN/ water (95:5, v/v) |
| SE19 | 1.0 equiv. Pamoic acid | // | // |
| SE20 | 1.0 equiv. Naphthalene- | Ongoing | Gel |
| | 2-sulfonic acid | | |
| SE21 | 1.0 equiv. Palmitic acid | // | // |
| SE22 | 1.0 equiv. Nicotinic acid | Ongoing | // |
| SE23 | 1.0 equiv. Vitamin C | Ongoing | Gel |
| SE24 | 1.0 equiv. Folic acid | // | // |
| SE25 | 1.0 equiv. Biotin | // | // |
| SE26 | Free form | Gel | Gel |

Explanation “//”: Not carried out.

### Example 11 Salt Metathesis

About 30 mg of batch 2 was dissolved into 0.215 ml of water. (Thin suspension) 1.05 equiv. of HCl (0.895 mL) was added into the glass vial. (Clear solution) Then 1.0 equiv. of Sodium lauryl sulfonate slowly charged into the clear solutions. (Clear solution)

0.3 mL of obtained clear solutions (Obtained from Slurry equilibration-2-2) were dispensed into 2 ml vials. 1.05 equiv. of HCl (0.895 mL) was added into the glass vial. (Clear solution) Then 1.0 equiv. of Sodium lauryl sulfonate slowly charged into the clear solutions. (Thin suspension) Obtained mixtures was stirred at 25° C. for 11 days.

Obtained suspensions were filtered through a 0.45 μm nylon membrane filter by centrifugation at 14,000 rpm. Solids were analyzed by XRPD.

The screening results were summarized in Table 11.

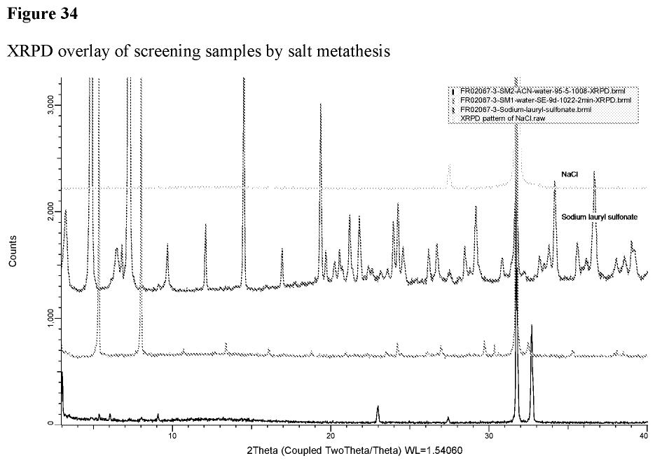

TABLE 11
Salt metathesis

| Exp. | | | |
|---|---|---|---|
| ID | Counter ions | Solvent | Comments |
| SM1 | Sodium lauryl | Water | 1.0 equiv. HCl → clear solution→ 1.0 equiv. of |
| | sulfonate | | sodium lauryl sulfonate → clear solution → |
| | | | equilibrated at 25° C. for 11 days → clear solution |
| | | | (brown) → placed at 5° C. for 56 days→ hazy |
| | | | solution (brown) → slow evaporation at ambient |
| | | | condition for 9 days: XRPD: NaCl + SLS FIG. 34 |
| SM2 | Sodium lauryl | ACN/water | 1.0 equiv. HCl → clear solution→ 1.0 equiv. of |
| | sulfonate | (95:5, v/v) | sodium lauryl sulfonate → thin suspension → |
| | | | equilibrated at 25° C. for 11 days → hazy solution + |
| | | | solids (brown) XRPD: NaCl + SLS FIG. 34 |

Explanation “AF”: Amorphous form.

### Example 12 Characterization of Cocrystal Hits

According to the cocrystal screening results (Table 3), obtained cocrystal hits will be further characterized by DSC, TGA, ¹H-NMR, IC, and KF.

### Example 13 Preparation of Cocrystal Candidates

Cocrystal candidates will be prepared to 500-1000 mg and evaluated in comparison of free form.

### Example 14 Solubility

Accurate 20 mg of free form will be weighed into a 20 mL vial. For cocrystal 1, X mg (equal to 20 mg free form) will be weighed into a 20 mL glass vial. 10 mL aqueous buffer will be added, respectively. These suspensions will be stirred at 37° C. with 400 rpm. These suspensions will be taken out at 2 hours and 24 hours, then centrifuged at 14,000 rpm for 5 min. The supernatants will be analyzed by HPLC. Solids obtained (wet cakes) will be characterized by XRPD.

### Instrumentation and Methods

Unless otherwise indicated, the following instrumentation and methods were used in the working examples as described herein.

X-ray Powder Diffractometer (XRPD)

| | |
|---|---|
| Instrument | Bruker D8 Advance |

Method 1 (About 10 min, for scale-up samples)

| | |
|---|---|
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary |
| | motorized slit 5.2 mm |
| Scan mode | Continuous scan |
| Scan type | Locked coupled |
| Step size | 0.02° |
| Time per step | 0.3 second per step |
| Scan range | 2° to 40° |
| Sample rotation speed | 15 rpm |
| Sample holder | Monocrystalline silicon, with cavity |

Method 2 (About 4 min, for evaluation samples)

| | |
|---|---|
| Detector | LYNXEYE_XE_T(1D mode) |
| Open angle | 2.94° |
| Radiation | Cu/K-Alpha1 (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary |
| | motorized slit 5.2 mm |
| Scan mode | Continuous scan |
| Scan type | Locked coupled |
| Step size | 0.02° |
| Time per step | 0.12 second per step |
| Scan range | 3° to 40° |
| Sample rotation speed | 15 rpm |
| Sample holder | Monocrystalline silicon, flat surface |

Method 3 (About 2 min, for cocrystal screening samples)

| | |
|---|---|
| Detector | LYNXEYE_XE_T(ID mode) |
| Open angle | 2.94° |
| Radiation | Cu/K-Alpha1 (λ = 1.5406 Å) |
| X-ray generator power | 40 kV, 40 mA |
| Primary beam path slits | Twin_Primary motorized slit 10.0 mm by sample length; |
| | SollerMount axial soller 2.5° |
| Secondary beam path slits | Detector OpticsMount soller slit 2.5°; Twin_Secondary motorized slit 5.2 mm |
| Scan mode | Continuous scan |
| Scan type | Locked coupled |
| Step size | 0.02° |
| Time per step | 0.06 second per step |
| Scan range | 3° to 40° |
| Sample rotation speed | 15 rpm |
| Sample holder | Monocrystalline silicon, flat surface |

Differential Scanning Calorimetric (DSC)

| | |
|---|---|
| Instrument | TA Discovery 2500 |
| Sample pan | Tzero pan and Tzero hermetic lid with a pin hole of 0.7 mm in diameter |
| Temperature range | 30 to 250° C. or before decomposition |
| Heating rate | 10° C./min |
| Nitrogen flow | 50 mL/min |
| Sample mass | About 0.5-2 mg |

Thermal Gravimetric Analysis (TGA)

| | |
|---|---|
| Instrument | Discovery 5500 or Q5000 |
| Sample pan | Aluminum, open |
| Start temperature | Ambient condition (below 35° C.) |
| Final temperature | 300° C. or abort next segment if weight <80% (w/w) (The weight loss |
| | of the compound is no more than 20% (w/w)) |
| Heating rate | 10° C./min |
| Nitrogen flow | Balance 10 mL/min; sample chamber 25 mL/min |
| Sample mass | About 2-10 mg |

Dynamic Vapor Sorption (DVS)

| | |
|---|---|
| Instrument | SPSadv-1 μ |
| Total gas flow | Max. 4000 mL/min |
| Oven temperature | 25° C. |
| Solvent | Water |
| Method | Cycle: 40-95-0-95-40% RH |
| | Stage Step: 10% |
| | Equilibrium: 240 min for each step |
| Sample mass | About 10-20 mg |

Karl Fischer (KF)

| | |
|---|---|
| Instrument | Mettler Toledo Coulometric KF Titrator C30 |
| Method | Coulometric |
| Sample mass | About mg |

Polarized Light Microscope (PLM)

| | |
|---|---|
| Instrument | Olympus BX53LED |
| Method | Crossed polarizer, silicone oil added |

Nuclear Magnetic Resonance (NMR)

| | |
|---|---|
| Instrument | Bruker Avance-AV 400M (for ¹H-NMR, ¹⁹F-NMR and ³¹P- NMR) |
| | Bruker Avance-III 400M (for ¹³C-NMR) |
| Frequency | 400 MHz |
| Probe | 5 mm PABBO BB/19F-1H/D Z-GRD Z108618/0406 (for |
| | ¹H-NMR, ¹⁹F-NMR and ³¹P-NMR) |
| | 5 mm PABBO BB-1H/D Z-GRD Z108618/0229 (for ¹³C NMR) |
| Number of scan | 8 |
| Temperature | 297.6 K |
| Relaxation delay | 1 second |

High Performance Liquid Chromatograph (HPLC)

| | |
|---|---|
| Instrument | SHIMADZU LC-20AD or Agilent 1260 infinityII Binary Pump |
| HPLC method | Wave length: |
| | Column: |
| | Detector: |
| | Column temperature: |
| | Flow rate: |
| | Mobile phase A: |
| | Mobile phase B: |
| | Diluent: |
| | Injection volume: |
| | Gradient: |
| | Time (min) Mobile Phase A (%) Mobile Phase B (%) |

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are encompassed by the following claims.

1-40. (canceled)

41. A (−) mesembrine composition comprising a solid form selected from the group consisting of:
a. a composition comprising (−) mesembrine and palmitic acid characterized by an XRPD pattern of FIG. 27 or by an XRPD pattern having peaks at 2theta 4.2° and 10.7° (±0.2 2theta);
b. a composition comprising (−) mesembrine and nicotinic acid and characterized by an XRPD pattern of FIG. 30;
c. a composition comprising (−) mesembrine and folic acid and characterized by an XRPD pattern of FIG. 31;
d. a composition comprising (−) mesembrine and biotin and characterized by an XRPD pattern of FIG. 32;
e. a composition comprising (−) mesembrine and pamoic acid and characterized by an XRPD pattern of FIG. 18, FIG. 19, FIG. 20, FIG. 21 or FIG. 26;
f. a composition comprising (−) mesembrine and L-lysine and characterized by an XRPD pattern of FIG. 5;
g. a composition comprising (−) mesembrine and glucose and characterized by an XRPD pattern of FIG. 1;
h. a composition comprising (−) mesembrine and glycine and characterized by an XRPD pattern of FIG. 9;
i. a composition comprising (−) mesembrine and nicotinamide and characterized by an XRPD pattern of FIG. 10;
j. a composition comprising (−) mesembrine and isonicotinamide and characterized by an XRPD pattern of FIG. 11 or FIGS. 33; and
k. a composition comprising (−) mesembrine and sodium lauryl sulfonate and characterized by an XRPD pattern of FIG. 34.

42. The composition of claim 41, wherein the composition comprises (−) mesembrine and palmitic acid and is characterized by an XRPD pattern of FIG. 27 or by an XRPD pattern having peaks at 2theta 4.2° and 10.7° (+0.2 2theta).

43. The composition of claim 41, wherein the composition comprises (−) mesembrine and nicotinic acid and is characterized by an XRPD pattern of FIG. 30.

44. The composition of claim 41, wherein the composition comprises (−) mesembrine and folic acid and is characterized by an XRPD pattern of FIG. 31.

45. The composition of claim 41, wherein the composition comprises (−) mesembrine and biotin and is characterized by an XRPD pattern of FIG. 32.

46. The composition of claim 41, wherein the composition comprises (−) mesembrine and pamoic acid and is characterized by an XRPD pattern of FIG. 18, FIG. 19, FIG. 20, FIG. 21 or FIG. 26.

47. The composition of claim 41, wherein the composition comprises (−) mesembrine and L-lysine and is characterized by an XRPD pattern of FIG. 5.

48. The composition of claim 41, wherein the composition comprises (−) mesembrine and glucose and is characterized by an XRPD pattern of FIG. 1.

49. The composition of claim 41, wherein the composition comprises (−) mesembrine and glycine and is characterized by an XRPD pattern of FIG. 9.

50. The composition of claim 41, wherein the composition comprises (−) mesembrine and nicotinamide and is characterized by an XRPD pattern of FIG. 10.

51. The composition of claim 41, wherein the composition comprises (−) mesembrine and isonicotinamide and is characterized by an XRPD pattern of FIG. 11 or FIG. 33.

52. The composition of claim 41, wherein the composition comprises (−) mesembrine and sodium lauryl sulfonate and is characterized by an XRPD pattern of FIG. 34.

53. A (−) mesembrine composition comprising (−) mesembrine in combination with 0.5-1.0 equivalent of a conformer, wherein the conformer is one or more compounds selected from the group consisting of valerenic acid, betulinic acid, 4-aminobenzoic acid, D-mannitol, L-proline, nicotinamide, isonicotinamide, glycine, n-methyl-D-glucamine, urea, L-phenylalanine, L-arginine, L-lysine, choline, glucose, nicotinic acid, palmitic acid, naphthalene-2-sulfonic acid, pamoic acid, saccharin, biotin, folic acid, and ascorbic acid, wherein the composition comprises one or more of the

54. The composition of claim 53, wherein the composition comprises a combination of the mesembrine and valerenic acid.

55. The composition of claim 53, wherein the composition comprises a combination of the mesembrine and betulinic acid.

56. The composition of claim 53, obtained by a process comprising the steps of (a) forming a solution of (−) mesembrine in a solvent, (b) combining the solution from step (a) with a coformer, and (c) obtaining a solid form from the composition of step (b).

57. A crystalline solid form composition of (−) mesembrine characterized by a XRPD spectra with one or more characteristic XRPD peaks of a XRPD spectrum selected from FIG. 1, FIG. 2, FIG. 5, FIG. 6, FIG. 7, FIG. 8, FIG. 9, FIG. 10, FIG. 14, FIG. 15, FIG. 16, FIG. 17, FIG. 18, FIG. 19, FIG. 21, FIG. 27, FIG. 30, FIG. 31, FIG. 32, FIG. 33 and FIG. 34.

58. The composition of claim 57, wherein the composition is a solid form of (−) mesembrine and pamoic acid characterized by a XRPD spectra with one or more characteristic XRPD peaks of the XRPD spectrum of FIG. 18.

59. The composition of claim 58, wherein composition is further characterized by the DSC thermogram of FIG. 22.

60. The composition of claim 41, wherein the composition is a pharmaceutical composition comprising an active pharmaceutical ingredient consisting of the (−) mesembrine solid form; and one or more pharmaceutically acceptable excipients.
